(19) United States
(12) Patent Application Publication
(10) Pub. No.: US 2026/0124476 A1
(43) Pub. Date: May 7, 2026

(54) PROTECTIVE HOOD

(71) Applicant: SAFRAN AEROSYSTEMS, PLAISIR (FR)

(72) Inventors: Thomas BRAUNLICH, MOISSY CRAMAYEL (FR); Jeremy GALLAND, MOISSY CRAMAYEL (FR); Fabien AESCHLIMANN, MOISSY CRAMAYEL (FR); Frederick JOUANNEAU, MOISSY CRAMAYEL (FR)

(73) Assignee: SAFRAN AEROSYSTEMS, PLAISIR (FR)

(21) Appl. No.: 19/382,786

(22) Filed: Nov. 7, 2025

Related U.S. Application Data
(63) Continuation of application No. PCT/FR2024/050587, filed on May 3, 2024.

(30) Foreign Application Priority Data
May 9, 2023 (FR) 23/04583

(51) Int. Cl. A62B 17/00; A61M 16/22; A62B 9/00; A62B 17/04; A62B 18/04; A62B 25/00

(52) U.S. Cl. CPC A62B 17/001; A62B 9/006; A62B 17/04; A62B 18/04; A62B 25/00; A61M 16/22

(57) ABSTRACT

A protective hood in a vacuum bag including a flexible shell that is folded when stored and when unfolded is pulled through an open base of the flexible shell and over the head of a user. The hood includes a visor and an articulated device rigidly attached to the open base to be placed around the neck of the user when the flexible shell is pulled over the head of the user. The articulated device includes at least two rigid portions connected by a joint that allows these two rigid portions to transition from folded, which inhibits the head of the user from entering the hood, to unfolded, which allows the head of the user to enter the hood. The hood includes a handle rigidly attached to the top of the flexible shell to be accessible to the user when the flexible shell is folded.

### CROSS-REFERENCE TO RELATED APPLICATIONS

This application is a continuation of International Application No. PCT/FR 2024/050587, filed on May 3, 2024, which claims priority to and the benefit of French Patent Application No. 23/04583 filed on May 9, 2023. The disclosures of the above applications are incorporated herein by reference.

### FIELD

The present disclosure relates to a protective hood, and a method for deploying same by a user, for example on board an aircraft.

### BACKGROUND

The statements in this section merely provide background information related to the present disclosure and may not constitute prior art.

Patent application EP2979561 discloses a hood comprising a sealed flexible shell intended to be put on the head of the user, the flexible shell being provided with a transparent window and comprising, in its lower part, a rigid base element of generally annular shape intended to be disposed around the neck of the user. The base element comprises a tubular oxygen tank provided with a calibrated outlet orifice emerging into the internal volume of the flexible shell. This type of device is used on board aircraft when the cabin atmosphere is polluted (depressurization, smoke, chemical agent, etc.). This equipment must in particular allow the crew members to combat the damage, rescue passengers and manage a possible evacuation of the aircraft.

### SUMMARY

This section provides a general summary of the disclosure and is not a comprehensive disclosure of its full scope or all of its features.

The present disclosure aims in particular to improve the deployment of a safety hood by a user.

The present disclosure thus relates to a method for deploying a protective hood by a user, this hood including: a flexible shell configured to be stored in a folded state and to be put, in an unfolded state, through an open base of the flexible shell, on the head of the user, an articulated device attached to the open base and configured to be positioned around the neck of the user when the flexible shell is put on the head of the user, the articulated device comprising at least two rigid parts connected by a joint allowing these two rigid parts to transition from a folded position inhibiting the head of the user from entering the hood to an unfolded position allowing the head of the user to enter the hood, a handle attached to the flexible shell, this handle being positioned on a top of the flexible shell in such a way as to be accessible to the user when the flexible shell is in the folded state, the method including the following: lifting the hood initially in the folded state, using the handle held by the user, in particular in the high position, so that the flexible shell can unfold by the action of the weight of at least the base of the flexible shell and the articulated device, to transition from the folded state to the unfolded state.

Thanks to the handle according to the present disclosure, which is advantageously positioned on the top of the flexible shell, it is possible for the user, for example crew members on board an aircraft, to deploy the hood by taking advantage of the gravity exerted on different components of the hood to unfold the flexible shell. These components which participate in the unfolding by gravity comprise the articulated device which, following the lifting of the hood, is located at the bottom of the flexible shell and participates in the unfolding action of the hood. The protective hood according to the present disclosure may be an operational hood ready to be used in actual operating conditions on board a vehicle, for example an aircraft, or alternatively, be a protective hood intended for training in a training center. In the case of a protective hood intended for training, this hood may be simpler than the hood for actual use, in the sense that the training hood may have fewer features, for example being devoid of an oxygen supply or being stored in the open air, without being positioned in a closed bag.

The present disclosure provides deployment of the hood simply by holding it vertically using the handle, without having to shake the hood suddenly.

According to one of the aspects of the present disclosure, the hood being initially stored in the folded state in a closed bag, in particular a vacuum bag, the method including the following:
opening the bag by the user to be able to remove the hood in the folded state, then unfolding the flexible shell by gravity using the handle.

According to one of the aspects of the present disclosure, the method includes the following: when the flexible shell is in the unfolded state, turning the hood over and acting on the articulated device so as to transition it from the folded position to the unfolded position.

According to one of the aspects of the present disclosure, the deployment method includes the following: spacing the two rigid parts apart to transition the articulated device into the unfolded position, so that the user can put on the hood.

According to one of the aspects of the present disclosure, the hood includes a timer configured to be triggered when the articulated device transitions from the folded position to the unfolded position.

According to one of the aspects of the present disclosure, the hood is configured to provide the user with at least one item of information regarding the use of the hood based on data provided by the timer, for example, this usage information being an alert indicating the end of an oxygen supply available in the articulated device when the timer has measured a predetermined duration, for example a duration of 15 minutes.

The fact that the triggering occurs when the articulated device transitions from the folded position to the unfolded position allows this triggering to be concomitant with the deployment of the hood. Thus, the presence of both the handle and the timer according to the present disclosure provides, in a synergistic manner, a rapid and efficient deployment of the hood to put it in an improved operating state.

The present disclosure also relates to a method for packaging a protective hood in a bag, in particular a vacuum bag, this hood including: a flexible shell configured to be stored in a folded state and to be put, in an unfolded state, through an open base of the flexible shell, on the head of the user, a visor attached, in a removable or non-removable manner, to the flexible shell and configured to allow the user to see through the hood when this hood is put on the head of the user, an articulated device attached to the open base and configured to be positioned around the neck of the user when the flexible shell is put on the head of the user, the articulated device comprising at least two rigid parts connected by a joint allowing these two rigid parts to transition from a folded position inhibiting the head of the user from entering the hood to an unfolded position allowing the head of the user to enter the hood, a handle attached to the flexible shell, this handle being positioned on a top of the flexible shell in such a way as to be accessible to the user when the flexible shell is in the folded state, the packaging method including the following: folding the hood so as to leave the handle visible and accessible when the hood is in the state folded, positioning the hood thus folded in the bag, closing the bag which then takes in particular the shape of a disk or any other shape.

According to one of the aspects of the present disclosure, when folding, the visor is positioned on the folded flexible shell so that folds of the flexible shell are located behind the visor.

In one example, the visor is positioned as flat as possible or while maintaining its initial curvature against the articulated device to inhibit the occurrence of cracks in the material when it remains constrained by folding for a long storage period which may be between 10 years and 15 years. Indeed, the hood can be stored in the bag for a storage period of up to 10 years to 15 years, a duration which corresponds to the maximum allowable duration before the expiration of the assembly.

The present disclosure also relates to a protective hood, in particular intended to be used in the deployment method as described above, including: a flexible shell configured to be stored in a folded state and to be put, in an unfolded state, through an open base of the flexible shell, on the head of the user, an articulated device attached to the open base and configured to be positioned around the neck of the user when the flexible shell is put on the head of the user, the articulated device comprising at least two rigid parts connected by a joint allowing these two rigid parts to transition from a folded position inhibiting the head of the user from entering the hood to an unfolded position allowing the head of the user to enter the hood, a handle attached to the flexible shell, this handle being positioned on a top of the flexible shell, in particular in such a way as to be accessible to the user when the flexible shell is in the folded state.

According to one of the aspects of the present disclosure, the handle is in the form of a flexible strip, in particular of a substantially rectangular shape.

Other strip-shaped forms can be provided, for example a trapezoid shape or a shape with a rounded edge.

According to one of the aspects of the present disclosure, the strip-shaped handle is solid, namely, being devoid of any orifices.

According to one of the aspects of the present disclosure, the handle is devoid of a loop.

These strip-shaped forms of the handle, different from a loop, make it possible to avoid dangerous risks of the handle catching on a part of the hood or the bag or the environment (for example a high element in an aircraft cabin) in which the hood is handled, risks which are likely to hinder the removal of the folded hood from the bag or the deployment of the hood or when the user moves with the hood on. In addition, the flat shape of the strip makes it possible to improve the volume taken up by the handle, in the folded position during storage.

According to one of the aspects of the present disclosure, the strip-shaped handle is fastened to the flexible shell by an edge of the strip, on an external face of this flexible shell. The fastening is obtained by glue, welding or by any other means. For example, the handle is heat-welded to the flexible shell.

According to one of the aspects of the present disclosure, the handle is made of self-extinguishing material, namely a material which can burn in a flame, but which extinguishes itself as soon as it is removed from it.

According to one of the aspects of the present disclosure, the handle has a color distinct from the color of the flexible shell.

According to one of the aspects of the present disclosure, the color of the handle is yellow or another distinctive color different from gray, in particular with a fluorescent effect.

Thus, the handle is easily and quickly identifiable by the user when the folded hood is taken out of the bag. It may be desired for the equipment setup time to be less than 15 seconds.

According to one of the aspects of the present disclosure, the articulated device has a mass of at least 50%, in particular at least 60% or 70%, of the total mass of the hood.

Thus, the articulated device, which is relatively heavy compared with the rest of the hood, reliably acts to unfold the hood by gravity.

According to one of the aspects of the present disclosure, the two parts of the articulated device are formed by two tubular, in particular curved or straight, portions connected by the joint, the two parts forming an open ring in the unfolded position.

It is understood that the term “annular” refers to a shape that partially surrounds the neck of the user, which may not be in the shape of a perfect ring.

According to one of the aspects of the present disclosure, the flexible shell is sealed and the articulated device is configured to provide a self-contained breathing function for the user wearing the hood. This applies to hoods intended for actual use, as opposed to hoods intended for training which may be simpler.

According to one of the aspects of the present disclosure, the articulated device comprises an oxygen tank, in particular of tubular shape, provided with an oxygen outlet orifice emerging into the internal volume of the flexible shell, and a cartridge for capturing CO2, for example lime, configured to adsorb the CO2 resulting from the respiration of the user. For example, the tubular portion that comprises the oxygen tank is curved, and the tubular portion that comprises the CO2 capture cartridge is straight. The material configured to adsorb CO2 may be porous.

In one variation, the flexible shell is permeable to air so that the user wearing the hood can breathe without respiratory assistance.

The hood, in this case, is for example a training hood, which may not have the functional equipment that must equip a protective hood for actual use. For example, the training hood can be equipped with an articulated device without a breathing function, while still allowing users to practice deploying the hood.

According to one of the aspects of the present disclosure, the hood includes an acoustic membrane.

According to one of the aspects of the present disclosure, the flexible shell mainly contains a Nomex® fabric, a flexible collar, in particular made of Neoprene®, a semi-rigid visor, in particular made of transparent polymer, and possibly an acoustic membrane made of rigid polymer material.

The present disclosure also relates to a protective hood, in particular intended to be used in the aforementioned deployment method, including: a flexible shell configured to be stored in a folded state and to be put, in an unfolded state, through an open base of the flexible shell, on the head of the user, an articulated device attached to the open base and configured to be positioned around the neck of the user when the flexible shell is put on the head of the user, the articulated device comprising at least at least two rigid parts connected by a joint allowing these two rigid parts to transition from a folded position inhibiting the head of the user from entering the hood to an unfolded position allowing the head of the user to enter the hood, a timer configured to be triggered when the articulated device transitions from the folded position to the unfolded position.

According to one of the aspects of the present disclosure, the two parts of the articulated device are formed by two tubular, in particular curved or straight, portions connected by the joint, and the timer is equipped with a trigger member configured to trigger the timer, this trigger member being linked, on the one hand, to the timer and, on the other hand, to the at least one of the parts of the articulated device so that the spacing between the two parts of the articulated device acts on the trigger member in order to trigger the timer.

According to one of the aspects of the present disclosure, the trigger member is connected to the timer in a removable manner so that the spacing of the two parts of the articulated device from its folded position to its unfolded position causes an extraction of the trigger member from the timer, extraction which results in a triggering of the timer.

According to one of the aspects of the present disclosure, the trigger member includes a trigger tab configured to be inserted into a slot of the timer.

According to one of the aspects of the present disclosure, the timer is configured to be in a deactivated mode while the tab is held in place.

According to one of the aspects of the present disclosure, the trigger tab is configured to act on a switch of the timer.

For example, the extraction of the tab allows the timer to be connected to an electrical power source, such as a battery.

According to one of the aspects of the present disclosure, the trigger tab is made of electrically insulating material, and is configured to keep open an electrical power supply circuit of the timer.

According to one of the aspects of the present disclosure, the extraction of this trigger tab causes the closure of the electrical power supply circuit of the timer, and the timer starts.

For example, the trigger tab is configured to be interposed between an electrical terminal of the timer and a cell, and removal of the trigger tab allows the cell to be brought into contact with the terminal, thereby closing the electrical power supply circuit.

According to one of the aspects of the present disclosure, the timer is part of an electronic card.

According to one of the aspects of the present disclosure, the electronic card is mounted on one of the two parts of the articulated device, in particular at a free end of the part formed by a CO2 capture cartridge, in particular a lime cartridge.

According to one of the aspects of the present disclosure, the timer, or the electronic card which defines the timer, is mounted on a cover of the CO2 capture cartridge.

This allows the number of parts to be reduced by eliminating a specific support for the timer.

Advantageously, the timer is positioned away from the oxygen container (on the other rigid part of the articulated device) which may include metal parts likely to present a risk of electric arcs.

According to one of the aspects of the present disclosure, the trigger member includes at least one link, in particular a wire, connecting the removable trigger tab and the opposite part of the articulated device.

According to one of the aspects of the present disclosure, the link, in particular the wire, is configured so that, when the articulated device transitions from its folded position to its unfolded position, this link is taut and pulls the removable tab to extract it from the timer.

According to one of the aspects of the present disclosure, the removable trigger tab comprises a double loop for the passage of the wire.

This double loop makes it possible to provide the greatest amount of material along the extraction axis of the tab.

According to one of the aspects of the present disclosure, the double loop made of a sufficiently flexible material of the tab, each with a notch entry, makes it easy to position the wire (or lace) in its notch. The assembly can be done with one hand without applying force in the direction of extraction of the tab. The elasticity of the plastic material is used to pass the lace without permanently deforming the tab.

According to one of the aspects of the present disclosure, a counter-notch is provided to inhibit the lace from becoming dislodged from the double loop.

According to one of the aspects of the present disclosure, the wire, or lace, has a sufficient length so that, when the hood is in the folded state, this wire, or lace, is relaxed. Thus, no tension is exerted on the trigger tab by the wire.

According to one of the aspects of the present disclosure, the removable trigger tab comprises a non-return member configured to oppose with a predetermined force the extraction of this tab from the slot of the timer.

According to one of the aspects of the present disclosure, the non-return member makes it possible, during assembly operations (for example folding and introduction into the bag and vacuuming), to eliminate the risk of applying a force to the tab which could trigger the timer involuntarily. The non-return member increases the force desired to remove the tab and thus inhibits involuntary triggering.

According to one of the aspects of the present disclosure, the non-return member comprises a protrusion.

According to one of the aspects of the present disclosure, the removable trigger tab is made from a plate, for example a plate made of plastic material, in particular cut with a water jet.

According to one of the aspects of the present disclosure, the hood is configured to provide the user with at least one item of information regarding the use of the hood based on data provided by the timer, for example, this usage information being an alert indicating the end of an oxygen supply available in the articulated device when the timer has measured a predetermined duration, for example a duration of 15 minutes.

According to one of the aspects of the present disclosure, the hood includes a light and/or sound alert device, for example a warning light, for example formed by one or several LEDs, configured to provide the user with information regarding the use of the hood.

According to one of the aspects of the present disclosure, the warning light is controlled by an electronic card which forms the timer, in particular to control the emission of light according to different modes.

According to one of the aspects of the present disclosure, the electronic card is configured to make the warning light blink for a first period (P1), for example one minute, then to keep the light continuously on for a second period (P2). The expiration of the first period (P1) corresponds in particular to the reaching of the regulatory usage time (Tcr) of the hood, which is, for example, 15 minutes. This regulatory usage time is determined by the manufacturer based on the availability of oxygen in the supply.

According to one of the aspects of the present disclosure, the electronic card is configured to switch off the warning light at the end of the second period (P2). The user must be able to remove the hood before the end of this period P2.

The present disclosure thus provides an effective alert to the user, so that he can manage the intervention time and remove the hood in time, when the oxygen supply of the hood is depleted.

Other alert sequences can of course be considered, for example using another light sequence and/or a sound sequence.

According to one of the aspects of the present disclosure, the timer is configured to trigger independently of the triggering of the oxygen supply.

The present disclosure also relates to a method for deploying a protective hood by a user, this hood including: a flexible shell configured to be stored in a folded state and to be put, in an unfolded state, through an open base of the flexible shell, on the head of the user, an articulated device attached to the open base and configured to be positioned around the neck of the user when the flexible shell is put on the head of the user, the articulated device comprising at least at least two rigid parts connected by a joint allowing these two rigid parts to transition from a folded position inhibiting the head of the user from entering the hood in an unfolded position allowing the head of the user to enter the hood, a timer configured to be triggered when the articulated device transitions from the folded position to the unfolded position, and the method including the following: spacing the articulated device so that it transitions from the folded position to the unfolded position, causing the timer to trigger.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

FIG. 1 is a schematic representation of a hood according to an exemplary implementation of the present disclosure, in the folded state;

FIG. 2 is a schematic representation of the hood of FIG. 1, in the unfolded state;

FIG. 3 is a schematic representation of the hood of FIG. 1, with the user spacing the articulated device in order to put on the hood;

FIG. 4 is an isolated schematic representation of the articulated device of the hood of FIG. 1;

FIG. 5 is a schematic representation of the hood of FIG. 1, put on the head of the user;

FIG. 6 is a schematic representation of a trigger member of the timer of the hood of FIG. 1;

FIG. 7 is a schematic representation of the hood of FIG. 1, stored in a bag; and

FIG. 8 is a schematic representation of an alert sequence associated with the timer of the hood of FIG. 1.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

The features, variants and different examples of the present disclosure may be combined with each other in various combinations, provided that they are not incompatible or mutually exclusive. In particular, variants of the present disclosure may be conceived comprising only a selection of features described below in isolation from the other described features, if this selection of features is sufficient to confer a technical advantage and/or to differentiate the present disclosure from the prior art.

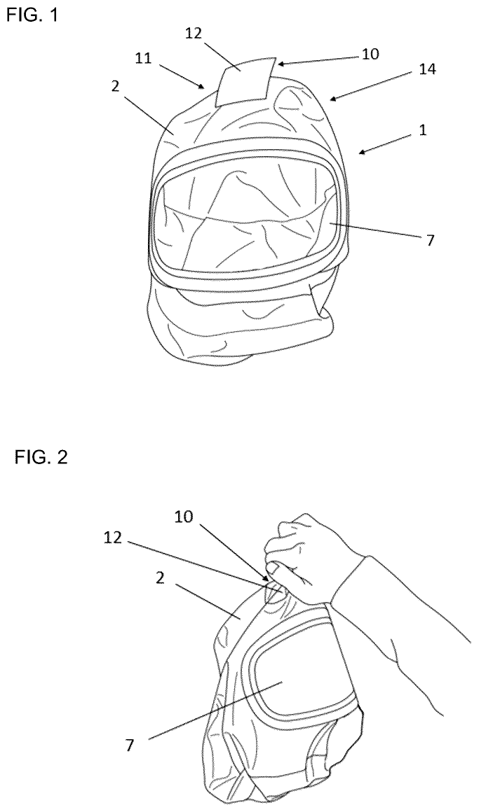

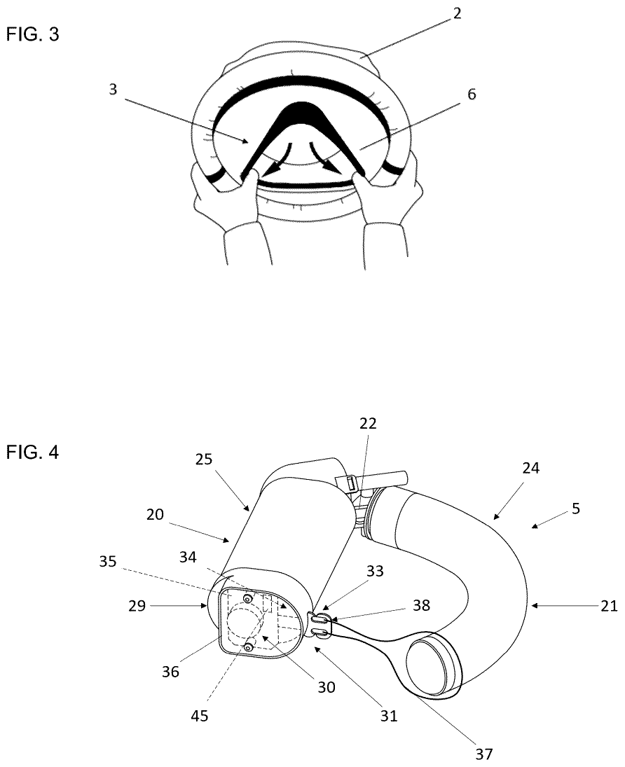

FIGS. 1 and 2 show a protective hood 1 including:
a flexible shell 2 configured to be stored in a folded state (state shown in FIG. 1) and to be put on, in an unfolded state (state shown in FIG. 2), through an open base 3 of the flexible shell 2, on the head of the user U,
an articulated device 5 (shown in isolation in FIG. 4) attached to the open base 3 and configured to be positioned around the neck of the user U when the flexible shell 2 is put on the head of the user.

In the described example, the flexible shell 2, which is airtight, mainly contains a Nomex® fabric and a flexible collar 6, in particular made of Neoprene®, forming the base 3.

The hood 1 also includes a semi-rigid visor 7, in particular made of transparent polymer, and possibly an acoustic membrane (not shown).

The hood 1 also includes a handle 10 attached to the flexible shell 2, this handle 10 being positioned on a top 11 of the flexible shell 2, in such a way as to be accessible to the user when the flexible shell 2 is in the folded state, as can be seen in FIG. 1.

In the described example, the handle 10 is in the form of a flexible strip 12, of substantially rectangular shape.

Other strip-shaped forms can be provided, for example a trapezoid shape or a shape with a rounded edge.

The band-shaped handle 10 is solid, namely it is devoid of any orifices. The handle 10 is devoid of a loop.

The strip-shaped handle 10 is fastened to the flexible shell 2 by an edge of the strip, on an external face 14 of this flexible shell 2. For example, the handle 10 is heat-welded to the flexible shell 2.

The handle 10 is made of self-extinguishing material, namely a material which can burn in a flame, but which extinguishes itself as soon as it is removed from it.

The handle 10 may or may not be made of the same material as the flexible shell. The handle 10 could, if desired, be made in one piece with the flexible shell 2, being for example an extension of the material of the flexible shell.

Advantageously, the handle 10 has a color distinct from the color of the flexible shell 2, here being fluorescent yellow.

Thus, the handle 10 is easily and very quickly identifiable by the user when he takes the folded hood 1 out of a bag. It may be desired for the equipment setup time to be less than 15 seconds.

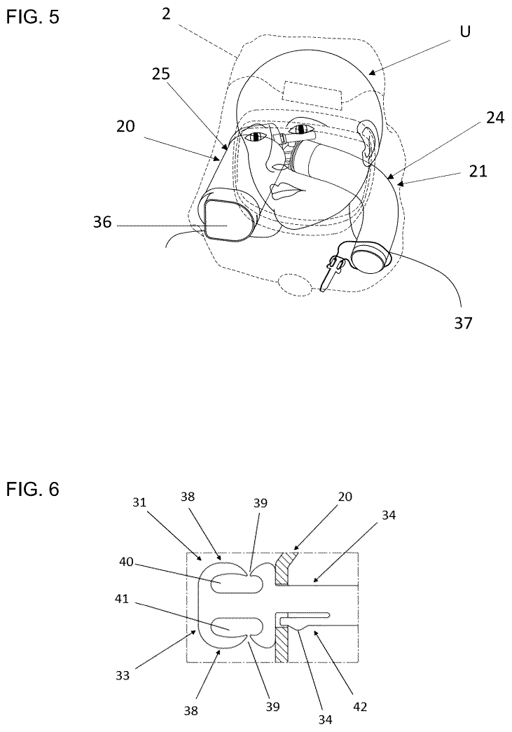

As can be clearly seen in FIG. 4, the articulated device 5 comprises two rigid parts 20 and 21 connected by a joint 22 allowing these two rigid parts 20 and 21 to transition from a folded position (state shown in FIG. 4) inhibiting the head of the user from entering the hood 1 to an unfolded position (state shown in FIG. 5) allowing the head of the user to enter the hood 1.

The articulated device 5 has a mass of at least 50%, in particular at least 60% or 70%, of the total mass of the hood 1.

Thus, the articulated device 5, relatively heavy compared with the rest of the hood, allows reliable action to unfold the hood 1 by gravity.

The two parts 20 and 21 of the articulated device are formed by two tubular portions connected by the joint 22 to form an open ring in the unfolded position.

The articulated device 5 is configured to provide a self-contained breathing function for the user having put on the hood 1. This is the case on hoods intended for actual use, unlike hoods intended for training which can be simpler.

Thus, the articulated device 5 comprises an oxygen tank 24 formed in the tubular part 21 and provided with an oxygen outlet orifice emerging into the internal volume of the flexible shell 2.

The tubular part 20 comprises a cartridge 25 for capturing CO2, for example lime, configured to adsorb the CO2 resulting from the breathing of the user.

In the described example, the tubular portion 21 which comprises the oxygen tank 24 is curved, and the tubular portion 20 which comprises the CO2 capture cartridge 25 is straight.

In the case of a training hood, which may not have the functional equipment that must equip a protective hood for real use, it can be equipped with an articulated device without a respiratory function, still allowing users to practice deploying the hood.

The tubular part 20 of the articulated device 5 includes, at its end 29 opposite to the joint 22, a timer 30 configured to be triggered when the articulated device 5 transitions from the folded position (FIG. 4) to the unfolded position (FIG. 5).

The timer 30 is equipped with a trigger member 31 configured to trigger the timer 30, this trigger member 31 being linked, on the one hand, to the timer 30 and, on the other hand, to the tubular part 21 of the articulated device 5 so that the spacing between the two parts 20 and 21 of the articulated device 5 acts on the trigger member 31 in order to trigger the timer 30.

The trigger member 31 is linked to the timer 30 in a removable manner so that the spacing of the two parts 20 and 21 of the articulated device 5 from its folded position to its unfolded position causes an extraction of the trigger member 31 from the timer 30, the extraction which results in a triggering of the timer which begins a countdown.

As can be clearly seen in FIGS. 4 and 6, the trigger member 31 includes a trigger tab 33 configured to be inserted into a slot 34 of the timer 30.

The timer 30 is configured to be in deactivated mode as long as the tab 33 is held in place.

For example, the extraction of the tab 33 makes it possible to connect the timer 30 to an electrical power source, such as a battery.

Alternatively, the trigger tab 33 is made of electrically insulating material, and is configured to keep open an electrical power supply circuit of the timer 30.

The extraction of this trigger tab 33 causes the closure of the electrical power supply circuit of the timer, and the timer starts.

The timer 30 is part of an electronic card 35.

For example, the timer 30, or the electronic card 35 which defines the timer, is mounted on a cover 36 of the CO2 capture cartridge 25.

Advantageously, the timer 30 is positioned away from the oxygen container 24 (on the other rigid part of the articulated device) which may include metal parts likely to present a risk of electric arcs.

The trigger member 31 includes at least one wire 37 connecting the removable trigger tab 33 and the opposite part 21 of the articulated device 5.

The wire 37 is configured so that, when the articulated device 5 is spaced from its folded position to its unfolded position, this wire 37 is taut and pulls the removable tab 33 to extract it from the slot 34 of the timer.

The removable trigger tab 33 comprises a double loop 38 for the passage of the wire 37.

This double loop 38 makes it possible to provide the greatest amount of material along the extraction axis of the tab 33.

The double loop 38 made of a sufficiently flexible material of the tab 33, each with a notch entry 39, makes it easy to position the wire 37 in the notch 40.

A notch 40 and a counter-notch 41 are provided to inhibit the lace from becoming dislodged from the double loop 38.

The wire 37 has a sufficient length so that, when the hood 1 is in the folded state, this wire 37 is relaxed (as can be seen in FIG. 4). Thus, no tension is exerted on the trigger tab by the wire 37.

The removable trigger tab 33 comprises a non-return member 42 configured to oppose with a predetermined force the extraction of this tab 33 from the slot 34 of the timer.

The non-return member 42, in the form of an elastic leg, makes it possible, during assembly operations (for example folding and introduction into the bag and vacuuming), to eliminate the risk of applying a force to the tab 33 likely to trigger the timer 30 involuntarily. The non-return member 42 increases the force to remove the tab 33 and thus inhibits involuntary triggering.

The non-return member comprises a protrusion 43 housed in the slot 34.

In the described example, the removable trigger tab 33 is made from a plate, for example a plate made of plastic material, in particular cut with a water jet.

The hood 1 is configured to provide the user with at least one item of information regarding the use of the hood based on data provided by the timer 30, this usage information being an alert indicating the end of an oxygen supply available in the articulated device 5 when the timer has measured a predetermined duration, for example a duration of 15 minutes.

The hood 1 includes a warning light 45, formed by one or several LEDs, configured to provide the user with information regarding the use of the hood. This light 45 is positioned on the electronic card 35.

The warning light 45 is controlled by the electronic card 35 to control the emission of light according to different modes.

For example, as illustrated in FIG. 8, following the triggering of the timer 30 at time T0 and after a period PS of, for example, 14 minutes, the electronic card 35 is configured to make the warning light 45 blink for a first period P1 which lasts, for example, one minute. The expiration of the first period P1 corresponds to the reaching of the time Tcr where the regulatory usage time of the hood expires, which is, for example, 15 minutes. This regulatory usage time is determined by the manufacturer based on the availability of oxygen in the supply.

Then the light 45 is kept continuously on for a second period P2, for example 3 minutes, then the electronic card 35 is configured to switch off the warning light 45 at the end of the second period P2. The user must be able to remove the hood before the end of this period P2.

The present disclosure thus provides an effective alert to the user, so that he can manage the intervention time and remove the hood in time, when the oxygen supply of the hood 1 is depleted.

Other alert sequences can of course be considered, for example using another light sequence and/or a sound sequence.

The timer 30 is configured to be triggered independently of the triggering of the oxygen supply.

We will now describe a method for packaging a protective hood 1 as described above, in a vacuum bag 50, the packaging method including the following: folding the hood 1 so as to leave the handle 10 visible and accessible when the hood 1 is in the folded state, positioning the hood 1 thus folded in the bag 50, when folding, the visor 7 is positioned on the folded flexible shell 2 (see FIG. 1) so that folds of the flexible shell are located behind the visor 7.

In one example, the visor 7 is positioned as flat as possible or while maintaining its initial curvature against the articulated device to inhibit the occurrence of cracks in the material when it remains constrained by folding for a long storage period which may be between 10 years and 15 years.

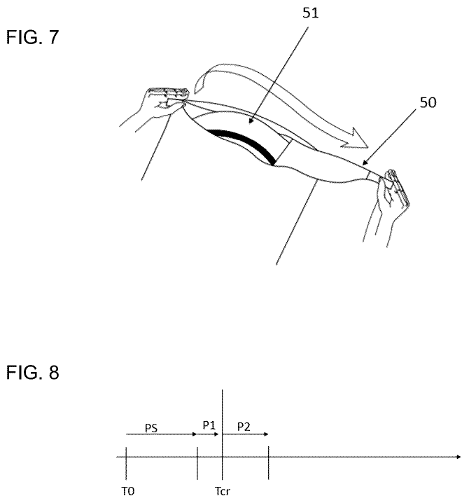

We will now describe a method for deploying a hood 1 as described above, initially stored under vacuum in the folded state in a bag 50, the method including the following: opening the bag 50 by the user U to be able to remove the hood 1 in the folded state (see FIG. 7), lifting the hood 1 initially in the folded state, using the handle 10 held by the user, in the high position, so that the flexible shell 2 can unfold by the action of the weight of the base of the flexible shell 2 and of the articulated device 5, to transition from the folded state to the unfolded state (see FIG. 2), when the flexible shell 2 is in the unfolded state, turning the hood 1 and separating the two rigid parts 20 and 21 from each other to transition the articulated device 5 in the unfolded position, so that the user can put on the hood 1.

Thanks to the present disclosure, it is thus possible to deploy the hood 1 simply by holding it vertically using the handle 10, without having to shake the hood suddenly.

The timer 30 is triggered when the articulated device 5 transitions from the folded position to the unfolded position, during deployment.

Unless otherwise expressly indicated herein, all numerical values indicating mechanical/thermal properties, compositional percentages, dimensions and/or tolerances, or other characteristics are to be understood as modified by the word “about” or “approximately” in describing the scope of the present disclosure. This modification is desired for various reasons including industrial practice, material, manufacturing, and assembly tolerances, and testing capability.

As used herein, the phrase at least one of A, B, and C should be construed to mean a logical (A OR B OR C), using a non-exclusive logical OR, and should not be construed to mean “at least one of A, at least one of B, and at least one of C.”

In this application, the term “controller” and/or “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components (e.g., op amp circuit integrator as part of the heat flux data module) that provide the described functionality; or a combination of some or all of the above, such as in a system-on-chip.

The term memory is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

The description of the disclosure is merely exemplary in nature and, thus, variations that do not depart from the substance of the disclosure are intended to be within the scope of the disclosure. Such variations are not to be regarded as a departure from the spirit and scope of the disclosure.

What is claimed is:

1. A method for deploying a protective hood by a user, comprising:
providing the protective hood, the protective hood including a flexible shell configured to be stored in a folded state and to be put, in an unfolded state, through an open base of the flexible shell, on a head of the user, an articulated device attached to the open base and configured to be positioned around a neck of the user when the flexible shell is put on the head of the user, the articulated device comprising at least two rigid parts connected by a joint allowing the at least two rigid parts to transition from a folded position that is configured to inhibit the head of the user from entering the protective hood to an unfolded position configured to allow the head of the user to enter the protective hood, and a handle attached to a top of the flexible shell, the handle configured to be accessible to the user when the flexible shell is in the folded state; and
lifting the protective hood initially in the folded state in a high position, such that the flexible shell unfolds by an action of a weight of at least the open base of the flexible shell and of the articulated device, to transition from the folded state to the unfolded state.

2. The method according to claim 1, wherein the protective hood is initially stored in the folded state in a closed vacuum bag and the method further comprises:
opening the vacuum bag by the user to remove the protective hood in the folded state; and
unfolding the flexible shell by gravity using the handle.

3. The method according to claim 2, further comprising:
when the flexible shell is in the unfolded state, turning the protective hood and acting on the articulated device so as to transition the protective hood from the folded state to the unfolded state.

4. The method according to claim 1, further comprising:
triggering a timer coupled to the protective hood based on the articulated device transitioning from the folded state to the unfolded state.

5. A method for packaging a protective hood in a vacuum bag, comprising:
providing the protective hood, the protective hood including a flexible shell configured to be stored in a folded state and to be put, in an unfolded state, through an open base of the flexible shell, on a head of a user, a visor attached, in a removable or non-removable manner, to the flexible shell and configured to allow the user to see through the protective hood when the protective hood is put on the head of the user, an articulated device attached to the open base and configured to be positioned around a neck of the user when the flexible shell is put on the head of the user, the articulated device comprising at least two rigid parts connected by a joint allowing these two rigid parts to transition from a folded position inhibiting the head of the user from entering the protective hood in an unfolded position allowing the head of the user to enter the protective hood, and a handle attached to the flexible shell, this handle being positioned on a top of the flexible shell in such a way as to be accessible to the user when the flexible shell is in the folded state;
folding the protective hood so as to leave the handle visible and accessible in the folded state;
positioning the folded protective hood in the vacuum bag; and
closing the vacuum bag to form a shape of a disk or any other shape.

6. The method according to claim 5, wherein, during the folding, the visor is positioned on the folded flexible shell so that folds of the flexible shell are located behind the visor.

7. A protective hood, comprising:
a flexible shell configured to be stored in a folded state and to be put, in an unfolded state, through an open base of the flexible shell, on a head of a user;
an articulated device attached to the open base and configured to be positioned around a neck of the user when the flexible shell is put on the head of the user, the articulated device including at least two rigid parts connected by a joint allowing these two rigid parts to transition from a folded position inhibiting the head of the user from entering the protective hood to an unfolded position allowing the head of the user to enter the protective hood; and
a handle attached to a top of the flexible shell, the handle configured to be accessible to the user when the flexible shell is in the folded state.

8. The protective hood according to claim 7, wherein the handle is a flexible strip having a rectangular shape, and having a color distinct from a color of the flexible shell.

9. The protective hood according to claim 7, further comprising
a timer configured to be triggered when the articulated device transitions from the folded position to the unfolded position.

10. The protective hood according to claim 9, wherein the two rigid parts of the articulated device are formed by two tubular portions connected by the joint, the two tubular portions are either curved or straight, and the timer is equipped with a trigger member configured to trigger the timer, the trigger member being linked, on a first end, to the timer and, on a second end, to at least one of the two rigid parts of the articulated device so that a spacing between the two rigid parts of the articulated device acts on the trigger member to trigger the timer.
